(19) United States
(12) Patent Application Publication
(10) Pub. No.: US 2020/0237717 A1
(43) Pub. Date: Jul. 30, 2020

(54) Treatment of Staphylococcal and Enterococcal Infections Using Substituted Nitrostyrene Compounds

(71) Applicant: Opal Biosciences Limited, South Melbourne, Victoria (AU)

(72) Inventors: Slade Jensen, North Epping, New South Wales (AU); Michael Radzieta, Campbelltown, New South Wales (AU)

(21) Appl. No.: 16/635,600

(22) PCT Filed: Jul. 30, 2018
(86) PCT No.: PCT/AU2018/050788

§ 371 Date: Jan. 31, 2020

(30) Foreign Application Priority Data
Aug. 1, 2017 (AU) 2017903033

(51) Int. Cl. A61K 31/36; A61K 31/4184; A61K 31/423; A61K 38/14; A61P 31/04

(52) U.S. Cl. CPC A61K 31/36; A61K 31/4184; A61P 31/04; A61K 38/14; A61K 31/423

(57) ABSTRACT

Disclosed are compositions comprising substituted nitrostyrene compounds, as well as methods for their manufacture. Also disclosed are methods of using these compounds in the treatment and/or amelioration of symptoms of bacterial infections such as those caused by Staphylococcus app. (including vancomycin-resistant strains of S. aureus) and Enterococcal spp.

### CROSS-REFERENCE TO RELATED APPLICATIONS

The present application claims priority to PCT Int. Pat. Appl. Publ. No. PCT/AU2018/050788 (WO 2019/023741 A1), filed Jul. 30, 2018 (nationalized), and to Australian Patent Application No. 2017903033, filed Aug. 1, 2017 (pending), the contents of each of which is specifically incorporated herein in its entirety by express reference thereto.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND OF THE INVENTION

### Field of the Invention

This disclosure relates generally to the use of substituted nitrostyrene compounds in the treatment of Staphylococcus aureus and Enterococcal infections. In particular aspects the present disclosure relates to the use of these compounds in the treatment of S. aureus infections, in which the bacteria have a level of vancomycin resistance.

### Description of Related Art

Antibiotics are the most widely used agents in the fight against pathogenic microorganisms. The antibiotic vancomycin is the leading member of the class of clinically important glycopeptide antibiotics used in the fight against life-threating and drug-resistant Gram-positive bacterial infections. In use since the 1950's, vancomycin is on the World Health Organisation's List of Essential Medicines (2013), a list of the most important medications needed in a basic health system.

However, since 1980 there has been an ever-increasing proportion of clinical bacterial isolates resistant to antibiotics. The steady increase of antimicrobial resistance is an evolutionary response to wide-spread antibiotic use. Antibiotic-resistant Gram-positive bacterial pathogens, including vancomycin-resistant enterococci (VRE), Clostridium difficile infection (CU) and Methicillin-resistant S. aureus (MRSA), have become serious concerns for global public health. In particular, increasing incidences of MRSA and VRE are seen.

The increasing emergence of vancomycin-resistant enterococci resulted in the development of guidelines for use of vancomycin, by the Centres for Disease Control's Hospital Infection Control Practices Advisory Committee (HICPAC). The National Institute of Allergy and Infectious Diseases (NIAID's) priority pathogens list includes Enterococcus faecium, Enterococcus faecalis (added 2014) and Staphylococcus aureus as “additional emerging infectious diseases/pathogens.”

MRSA is particularly problematic clinically as it is multi-drug resistant and refractory to many common clinic antibiotics. MRSA is considered to be any strain of Staphylococcus aureus that has developed, through the process of natural selection, resistance to beta-lactam antibiotics, which include the penicillins (methicillin, dicloxacillin, nafcillin, oxacillin, etc.) and the cephalosporins. The evolution of such resistance does not cause the organism to be more intrinsically virulent than strains of S. aureus that have no antibiotic resistance, but resistance does make MRSA infection more difficult to treat with standard types of antibiotics and thus more dangerous.

Vancomycin, formerly a gold standard treatment for MRSA, has become increasingly ineffective because of the emergence of vancomycin-resistant and vancomycin-intermediate Staphylococcus aureus (VRSA and VISA). An MRSA isolate with decreased susceptibility to vancomycin was first reported in Japan in 1997 (Hiramatsu, K et al., 1997). The isolate had only a modestly increased minimum inhibitory concentration (MIC) value for vancomycin, in the range of 3-8 μg/mL, and became known as vancomycin intermediate-resistant S. aureus (VISA). VISA began to be reported with increasing frequency among MRSA isolates identified all over the world. In spite of their moderate increase in MIC value, vancomycin treatment of infections with VISA isolates often ended in treatment failure (See e.g., Linares, J, 2001; Fridkin, S K et al., 2003). In 2002, the first vancomycin-resistant S. aureus (VRSA) strain (with a vancomycin MIC value greater than 100 μg/mL) was reported in the United States (Sievert, D M et al., 2008).

Heteroresistant subpopulations of vancomycin-susceptible S. aureus (hVISA) were first described in 1997 (Hiramatsu, K et al., 1997), shortly after the initial description of vancomycin intermediate susceptible strains (VISA). hVISA have minimum inhibitory concentrations (MICs) in the intermediately susceptible range and are considered to likely represent a step on the path to the development of a fully VISA population. While high-level vancomycin resistance has been rarely reported, VISA and hVISA are more common and are associated with clinical failure. Clearly the use of vancomycin therapy against such isolates would be impossible. As a consequence, clinicians are continually being challenged by infections caused by S. aureus and the treatment of suspected S. aureus and other vancomycin-resistant bacterial infections is becoming increasingly more complicated.

As the prevalence of vancomycin-resistant bacterial strains increases, new antibiotics with the longevity and dependability of vancomycin will be required to contain their impact. This need is arising at the same time that antibiotic discovery efforts are being discontinued at most major pharmaceutical companies. The reasons for this decline in antibiotic development are largely economic, resulting from a combination of patient short term use, the restricted use of new antibiotics with activity against resistant bacteria, and the increased regulatory criteria for approval.

Protection from infectious disease therefore relies not only on the effective management of current antimicrobial agents, but also on the development of new classes of compounds, and the identification of new microbial targets in order to address the serious problems caused by the emergence of drug-resistant human and animal pathogens (See e.g., Boucher, H W, 2010). Unfortunately, developmental drugs have a low probability of reaching and completing clinical development (See e.g., Payne, D J et al., 2007) so the need to identify new targets and agents remains perpetually urgent. In particular, there is an unmet need for effective treatment and/or prevention of antibiotic resistant S. aureus, E. faecalis and E. faecium associated infections, including those caused by MRSA, VRE, VISA and VRSA.

The antimicrobial properties of several benzyl nitroethenes have been reported (Nicoletti, G et al., 2013; Schales, O and Graefe, H A, 1952; Worthen, L R and Bond, H W, 1970, Milhazes, N et al., 2006). Nicoletti et al. have investigated the structure-activity relationships (SAR) of 23 benzyl nitroalkenes against a panel of clinically significant bacterial (Vincent, C et al., 2000) and fungal species (Worthen, L R and Bond, H W, 1970). The SAR study showed the importance of the nitroethenyl and nitropropenyl side chain to anti-microbial activity, proposed by Park and Pei to be essential for inhibition of protein tyrosine phosphatases (PTP), with the nitropropenyl substituent being the most active (Park, J and Pei, D, 2004). The 23 compounds showed broad antimicrobial activity that differed across species, with greatest activity against Gram-positive bacteria and fungi and least against enteric Gram-negative rods. One of the most active compounds was a substituted nitrostyrene compound and a tyrosine mimetic, 3,4-methylenedioxy-β-methyl-β-nitrostyrene. It is broadly microbicidal to many Gram-negative and most Gram-positive bacterial species as well as many fungal species and is active against strict anaerobes and intracellular pathogens including Mycobacterium tuberculosis and Plasmodium falciparum (See e.g., Nicoletti, G et al., 2013; Denisenko, P P, 2002; White, K S, 2008). It has low oral toxicity for mice and chickens and is poorly absorbed from the intestinal tract (See e.g., Nicoletti, G et al., 2013; Nicoletti, A et al., 2004).

### SUMMARY OF THE INVENTION

In a first aspect, the present invention provides a method of treating S. aureus infection in a patient wherein the S. aureus has at least partial resistance to vancomycin, the method comprising administering to the patent an effective amount of a compound of formula (I) or a pharmaceutically acceptable salt or derivative thereof:

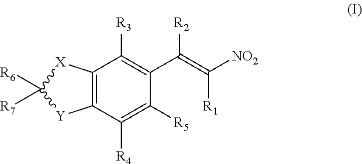

in which X and Y are either the same or different and are each a heteroatom selected from the group consisting of 0, N, and S;

[symbol] is a double or single bond depending on the heteroatoms X and Y;

R₁ to R₅ are either the same or different and selected from hydrogen, alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, acylthio, acylthio or phosphorus-containing compounds; and R₆ and R₇ are either the same or different, and selected from hydrogen, alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, arylthio, acylthio or phosphorus-containing compounds, or one of R₆ and R₇ are absent when there is a double bond present.

In an embodiment, in the compound of formula I, X and Y are either the same or different and selected from 0 and N, more preferably both X and Y are oxygen.

In an embodiment, in the compound of formula I R₁ and R₂ are either the same or different and selected from hydrogen, hydroxy, halogen or optionally substituted C₁₋₆ alkyl.

In the compound of formula I, R₃ to R₅ are preferably either the same or different and selected from hydrogen, hydroxy, halogen, nitro, C₁₋₆ alkoxy or optionally substituted C₁₋₆ alkyl. Preferably halogen is chlorine or bromine.

The E isomer of the compounds of formula I is preferred.

In an embodiment, are compounds of the formula I in which X, Y, [symbol] , R₆ and R₇ are as defined above; R₁ and R₂ are either the same or different and selected from hydrogen, hydroxy, Cl, Br and C₁₋₄ alkyl; and R₃ to R₅ are either the same or different and selected from hydrogen, hydroxy, Cl, Br, nitro, C₁₋₄ alkoxy or C₁₋₄ alkyl.

Specific examples of the compounds of the compounds of formula I are as follows:
X and Y are O, R₁ is methyl and R₂ and R₃ are hydrogen (3,4-methylenedioxy-β-methyl-β-nitrostyrene) (BDM-I)

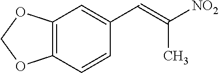

X and Y are 0 and R₁ to R₃ are hydrogen (3,4-methylenedioxy-β-nitrostyrene)

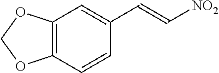 [SMILES: O=[N+]([O-])/C=C/c1ccc2c(c1)OCO2]

X is N, Y is NH, R₁ is methyl and R₂ and R₃ are hydrogen (benzimidazole-5-β-nitropropylene)

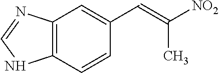

X is N, Y is NH, R₁ is hydrogen, R₂ is methyl and R₃ is absent (2-methyl benzimidazole-5-β-nitroethylene)

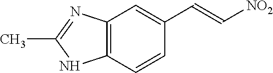

X is O, Y is N, R₁ and R₂ are hydrogen and R₃ is absent (benzoxazole-5-β-nitroethylene)

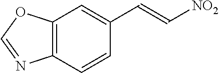 [SMILES: O=[N+]([O-])/C=C/c1ccc2ncoc2c1]

X is N, Y is O, R₁ and R₂ are methyl and R₃ is absent (2-methyl benzoxazole-5-β-nitropropylene)

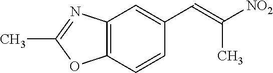 [SMILES: C/C(=C\c1ccc2oc(C)nc2c1)[N+](=O)[O-]]

In a preferred embodiment the compound is X and Y are O, R₁ is methyl, and R₂ and R₃ are hydrogen (3,4-methylenedioxy-β-methyl-β-nitrostyrene) (BDM-I)

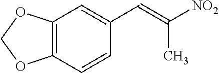

In a second aspect the present invention provides a method of treating S. aureus infection in a patient wherein the infection has failed to resolve following vancomycin treatment, the method comprising administering to the patient a compound of formula (I) or a pharmaceutically-acceptable salt or derivative thereof as defined herein.

In particular embodiments, the compound is BDM-I.

In a third aspect, the present invention provides a method of treating Enterococcal infection in a patient, the method comprising administering to the patient vancomycin or a derivative thereof and a compound of formula (I) or a pharmaceutically-acceptable salt or derivative thereof as defined herein.

In particular embodiments, the Enterococcus is vanB VRE, and the compound of Formula I is BDM-I. In certain embodiments, the Enterococcus is E. faecalis or E. faecium.

In a further aspect the present invention provides a composition comprising a combination of vancomycin or a derivative thereof and a compound of formula I or a pharmaceutically-acceptable salt or derivative thereof as defined herein.

In an embodiment, the composition is a pharmaceutical composition comprising one or more pharmaceutically-acceptable carriers, diluents and/or excipients.

In embodiments, the S. aureus is MRSA, VRSA, VISA or hVISA.

In an embodiment, the compound of formula I or a pharmaceutically-acceptable salt or a derivative thereof defined above and vancomycin or a derivative thereof are present in the composition in amounts which together are sufficient to treat a disease or condition in a subject. In an embodiment, the compound of formula I or a pharmaceutically-acceptable salt or a derivative thereof defined above and vancomycin or a derivative thereof are present in the composition in synergistically-effective amounts. In an embodiment, the compound of formula I or a pharmaceutically acceptable salt or derivative thereof as defined above and/or vancomycin or a derivative thereof are present in the composition in an amount which is less than the minimum inhibitory concentration (MIC) of the agent when used independently.

### BRIEF DESCRIPTION OF THE DRAWINGS

The following drawings form part of the present specification and are included to demonstrate certain aspects of the present invention:

FIG. 1 shows an average BDM-I MIC of triplicate Sa375 cultures serially passaged in the presence of BDM-I over a period of 110 days. Samples were collected for MIC testing when growth could (or could not) be maintained in higher BDM-I concentrations. Error bars denote standard deviation; and

FIG. 2 shows the results of BDM-I and vancomycin checkerboard assays (via broth micro dilution method) for vanB VRE clinical isolates. Individual MICs are circled and indicated as “red,” while the numbers in grey boxes represent FICi values for different BDM-I and vancomycin combinations. X denotes growth and each checkerboard assay was performed in triplicate (identical result each time). Note that Enterococcus faecium isolates are considered vancomycin sensitive if the MIC is ≤4 mcg/mL.

### DESCRIPTION OF ILLUSTRATIVE EMBODIMENTS

The terminology used herein is for the purpose of describing particular embodiments only and is not intended to be limiting. Unless defined otherwise, all technical and scientific terms used herein have the same meanings as commonly understood by one of ordinary skill in the art to which the present disclosure belongs. Any materials and methods similar or equivalent to those described herein can be used to practice the present invention.

Throughout this specification, unless the context requires otherwise, the word “comprise,” or variations such as “comprises,” or “comprising,” will be understood to imply the inclusion of a stated element or integer or group of elements or integers but not the exclusion of any other element or integer or group of elements or integers.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the singular forms “a”, “an,” and “the” include plural aspects unless the context clearly dictates otherwise. Thus, for example, reference to “a bacteria” includes a single bacterium, as well as two or more bacteria; reference to “an organism” includes one organism, as well as two or more organisms; and so forth.

The present disclosure is predicated in part, on the inventors' findings that there is an inverse relationship between MICs for BDM-I and vancomycin MICs in methicillin-resistant S. aureus. This “see-saw” effect indicates an advantage for the use of BDM-I in the treatment MRSA infections where there is at least a level of vancomycin resistance. The present inventors have also noted a synergy between BDM-I and vancomycin against Enterococcus.

In a first aspect, the present invention provides a method of treating S. aureus infection in a patient wherein the S. aureus has at least partial resistance to vancomycin, the method comprising administering to the patent an effective amount of a compound of formula (I) or a pharmaceutically acceptable salt or derivative thereof.

In a second aspect the present invention provides a method of treating S. aureus infection in a patient wherein the infection has failed to resolve following vancomycin treatment, the method comprising administering to the patient a compound of formula (I) or a pharmaceutically acceptable salt or derivative thereof as defined herein.

In a third aspect the present invention provides a method of treating Enterococcal infection in a patient, the method comprising administering to the patient vancomycin or a derivative thereof and a compound of formula (I) or a pharmaceutically-acceptable salt or derivative thereof as defined herein.

In particular embodiments, the Enterococcus is vanB VRE and the compound of Formula I is BDM-I. In certain embodiments, the Enterococcus is E. faecalis or E. faecium.

In a fourth aspect, the present invention provides a composition comprising a combination of vancomycin or a derivative thereof and a compound of formula I or a pharmaceutically-acceptable salt or derivative thereof as defined herein.

In a fifth aspect, the present invention provides the use of a compound of formula (I) or a pharmaceutically acceptable salt or derivative thereof in the preparation of a medicament for use in treatment of S. aureus infection in a patient wherein the S. aureus has at least partial resistance to vancomycin.

In a sixth aspect, the present invention provides the use of a compound of formula (I) or a pharmaceutically acceptable salt or derivative thereof in the preparation of a medicament for use in treatment of S aureus infection in a patient wherein the infection has failed to resolve following vancomycin treatment.

In a seventh aspect, the present invention provides the present invention provides the use of a compound of formula (I) or a pharmaceutically acceptable salt or derivative thereof in the preparation of a medicament for use in treatment of Enterococcal infection in a patient, wherein the medicament is administered in combination with vancomycin or a derivative thereof.

In particular embodiments, the compound is BDM-I.

In a compound of formula (I), the term “heteroatom” denotes O, N or S.

The term “halogen” refers to fluorine, chlorine, bromine and iodine, preferably chlorine and bromine.

The term “alkoxy” is used herein in its broadest sense and refers to straight chain, branched chain or cyclic oxy-containing radicals each having alkyl portions, preferably C₁₋₆ alkyl, more preferably C₁₋₄ alkyl. Examples of such alkoxy groups are methoxy, ethoxy, propoxy, butoxy and t-butoxy.

The terms “C₁₋₄ alkyl” or “C₁₋₆ alkyl” refer to straight chain, branched chain or cyclic hydrocarbon groups having from 1 to 6 carbon atoms. Illustrative of such alkyl groups are methyl, ethyl, propyl, isopropyl, butyl, isobutyl, sec-butyl, tert-butyl, pentyl, neopentyl, hexyl, cyclopropyl, cyclobutyl, cyclopentyl or cyclohexyl.

The salts of the compound of formula I are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

As used herein, the term “pharmaceutically-acceptable salt” refers to a compound formulated from a base compound which achieves substantially the same pharmaceutical effect as the base compound.

In addition, some of the compounds of formula (I) as defined herein may form solvates with water or common organic solvents. Such solvates are encompassed within the scope of the invention.

The term “derivatives,” in relation to the compound of formula I, includes, but is not limited to, ether derivatives, acid derivatives, amide derivatives, ester derivatives and the like. In addition, this invention further includes methods utilizing hydrates of the compound. The term “hydrate” includes but is not limited to hemihydrate, monohydrate, dihydrate, trihydrate and the like.

In an embodiment, in the compound of formula I, X and Y are O, R₁ is methyl and R₂ and R₃ are hydrogen (3,4-methylenedioxy-β-methyl-β-nitrostyrene) and the compound has the following structure:

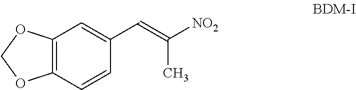

Vancomycin is well known in the art as an antibiotic used to prevent or inhibit the growth of Gram-positive bacteria. In particular, vancomycin is used to treat or prevent bacterial infections caused by Gram-positive bacteria.

The term “vancomycin derivative” as used herein refers to a compound having a structure derived from vancomycin, which exhibits the same or substantially similar biological activity and physicochemical properties as vancomycin. Examples include, but are not limited to, salts, esters, amides, salts of esters or amides, and N-oxides of vancomycin. In the context of the disclosures herein, reference to the biological activity of vancomycin is reference to its inhibitory or biocidal activity on Gram-positive bacterial growth.

The present inventors have found that an increasing thickness of the bacterial cell wall which accompanies an increase in vancomycin MIC leads to increasing BDM-I sensitivity. In line with this finding is a demonstration that increasing BDM-I MIC result in decreasing cell wall thickness which correlates with decreasing vancomycin MIC. The present have also noted a synergy between BDM-I and vancomycin in relation to sensitivity of vanB vancomycin-resistant Enterococci.

Accordingly, in a further aspect there is provided a method of increasing the biocidal activity of vancomycin or a derivative thereof against Enterococcus, the method comprising contacting the Enterococcus with the vancomycin or a derivative thereof, together with a compound of formula I or a pharmaceutically-acceptable salt or derivative thereof as defined herein.

In an embodiment, the vancomycin or a derivative thereof is used or administered together with a compound of formula I or a pharmaceutically-acceptable salt or derivative thereof as defined herein to inhibit or prevent antibiotic-resistant bacterial growth or treat, inhibit or prevent an antibiotic-resistant bacterial infection.

In an embodiment, the antibiotic-resistant bacteria are vancomycin-resistant bacteria.

It should be understood that reference to “vancomycin-resistant bacteria” includes vancomycin-resistant Gram-positive strains of bacteria that are both completely resistant to inhibition or prevention of growth by vancomycin or which require high levels of vancomycin to prevent or inhibit bacterial growth.

As is known in the art there are three classes of vancomycin-resistant S. aureus that differ in vancomycin susceptibility. Vancomycin-intermediate S. aureus (VISA), heterogeneous vancomycin-intermediate S. aureus (hVISA), and high-level vancomycin-resistant S. aureus (VRSA). VISA has also been termed GISA (glycopeptide-intermediate S. aureus), indicating resistance to all glycopeptide antibiotics. MRSA also demonstrates both intermediate and full-resistance to vancomycin. As used herein, reference to vancomycin-resistant S. aureus should be understood as reference to all strains of S. aureus which demonstrate a level of resistance to vancomycin, including VISA, hVISA, VRSA, GISA and MRSA.

Without limiting the present invention to any one theory or mode of application, the present inventor's have found that the combination of the compound of formula I or a pharmaceutically acceptable salt or derivative thereof and vancomycin, significantly decreased bacterial growth of vanB VRE in comparison to exposure of these antibiotic-resistant bacterial strains to each compound independently. Accordingly, in an embodiment disclosed herein, VRE comprises the vanB resistance gene.

In an embodiment, the vancomycin or a derivative thereof and a compound of formula I or a pharmaceutically-acceptable salt or derivative thereof are in the form of a pharmaceutical composition.

Accordingly, in another aspect of the present invention, there is provided a pharmaceutical composition for use in a method of reducing bacterial growth, or for preventing, inhibiting or treating a bacterial infection in a subject, the composition comprising vancomycin or a derivative thereof and a compound of formula I or a pharmaceutically-acceptable salt or derivative thereof as defined herein, together with one or more pharmaceutically acceptable carriers, diluents or excipients.

A “pharmaceutically acceptable carrier, diluent and/or excipient” as used herein, is a pharmaceutically acceptable solvent, suspending agent or vehicle for delivering the composition enabled herein to the subject. The carrier may be liquid or solid and is selected with the planned manner of administration in mind. Each carrier must be pharmaceutically “acceptable” in the sense of being compatible with other ingredients of the composition and non-injurious to the subject.

The vancomycin or a derivative thereof and the compound of formula I or a pharmaceutically acceptable salt or derivative thereof are present in the combination or composition in synergistically effective amounts.

The expression “synergistically effective amounts” of vancomycin or a derivative thereof, and the compound of formula (I) or a pharmaceutically acceptable salt or derivative thereof, refers to an amount of each component of the combination or composition which is effective in producing more than the additive effect of each component on inhibiting or preventing bacterial growth. The person of ordinary skill in the art would be well aware of particular methods which can be used to determine the effects of two compounds in an antimicrobial assay. For example, checkerboard, fractional inhibitory concentration (FIC) and time-kill analysis methods are commonly used. Synergy can be defined in terms of the fractional inhibitory concentration (FIC) index, which is the sum of the FIC's for the individual drugs used in the combination, as described by Sande et al., p. 1080-1105 in A. Goodman et al., ed., “The Pharmacological Basis of Therapeutics,” MacMillan Publishing Co., Inc., New York (1980). Under a strict scientific, and preferred, definition synergy is defined by an FIC index of less than 0.5, i.e., when 50% inhibition results from a combination of one-fourth or less of the concentration of each drug required to elicit the same effect if used individually (i.e., the minimal inhibiting concentration (MIC) of each drug). An FIC index of 0.5 under this strict definition defines an additive response. Under a broader definition used for purposes herein synergistically effective amounts are defined by an FIC index of less than 1.0, i.e., when 50% inhibition results from a combination of one-half or less of the MIC of each drug. An FIC index of 1.0 under this broader definition defines an additive response. Under this test, isobolograms may be prepared from the dose response curves for various combinations of vancomycin or a derivative thereof and compound of formula (I) or a pharmaceutically acceptable salt or derivative thereof on bacterial growth, with synergy indicated by points below the line which line connects the FIC index of 1 for vancomycin or a derivative thereof with the FIC index of 1 for compound of formula (I) or a pharmaceutically acceptable salt or derivative thereof. This standard allows one to determine the MIC's for the combinations tested, so as to provide the MIC of each component needed to achieve a synergistic mixture. The exact amounts will depend, for example, on the particular Gram-positive bacterial strain and the structure of the compound of formula (I) or a pharmaceutically acceptable salt or derivative thereof or vancomycin or derivative thereof employed.

The practitioner skilled in the art will recognize that actual dosages may vary within a relatively wide range because of inherent limitations in the above calculations and will be able, according to available skill in the art, to determine more precisely those amounts of the components which will be effective in producing synergistic results.

Reference to “synergy” as defined herein should also be understood as reference to the anti-bacterial activity of the composition comprising a combination of vancomycin or a derivative thereof and the compound of formula I or a pharmaceutically acceptable salt or a derivative thereof. Without limiting the present invention to any one theory or mode of action, the synergistic anti-bacterial activity includes an enhancement or potentiation of the anti-bacterial activity of vancomycin. The synergistic activity also includes a reduction in the dosage or MIC of each agent required to inhibit, reduce or prevent Gram-positive bacterial growth.

Suitable methods for determining the MIC of a particular antimicrobial agent would be well known to persons skilled in the art. In an illustrative example, the MIC of both vancomycin or the compound of formula I or a derivative or pharmaceutically salt thereof, can be determined by agar dilution method using CLSI guidelines (Performance standards for antimicrobial susceptibility testing, 17th informational supplement (M100-517) Wayne, Pa.: Clinical and Laboratory Standards Institute; 2007. Clinical and Laboratory Standards Institute). However, it will be understood by persons skilled in the art that any suitable method for determining MIC can be used.

Using the Clinical and Laboratory Standards Institute (CLSI) and the United States Food and Drug Administration (FDA) interpretive criteria, S. aureus is considered Vancomycin susceptible at an MIC of ≤2 mcg/mL and vancomycin intermediate if the MIC is 4 to 8 mcg/mL. The European Committee to Harmonize Antimicrobial Breakpoints (EUCAST) considers S. aureus resistant to vancomycin if the MIC is ≤2 mcg/mL.

E. faecium isolates are considered vancomycin sensitive if the MIC is mcg/mL.

The specific dosage “amount” will, obviously, vary with such factors as the particular condition being treated, the physical condition of the subject, the type of subject being treated, the duration of the treatment, the nature of concurrent therapy (if any), and the specific formulations employed and the structure of the compound of formula (I) or its derivatives. The dosage amounts of vancomycin which may be utilised are well known in the art.

The composition of the present invention may additionally be combined with other medicaments to provide an operative combination. It is intended to include any chemically compatible combination of pharmaceutically active agents, as long as the combination does not eliminate the activity of the combination or composition described herein. A non-limiting example of other agents is antimicrobial agents.

It will be appreciated that the combination or composition described herein and the other medicament may be administered separately, sequentially or simultaneously. Other medicaments which may be used when treating microbial infections include other anti-infective agents such as antibiotics.

The terms “administer”, “administering” or “administration” in reference to a combination or, composition as described herein means introducing the combination or composition into the system of a subject in need of treatment. When a combination or composition described herein is provided in combination with one or more other active agents, “administration” and its variants are each understood to include concurrent and/or sequential introduction of the compound and the other active agents.

In particular embodiments of any of the methods of the invention, the two components of the combination therapy are administered within 10 days of each other, within five days of each other, within twenty-four hours of each other, or simultaneously. The compounds may be formulated together as a single composition or may be formulated and administered separately. The duration of the treatment depends on the type of bacterial infection being treated, the age and condition of the subject, the stage and type of the subject's disease, and how the subject responds to the treatment. Additionally, a person having a greater risk of developing a bacterial infection (e.g., a person who is undergoing a surgical procedure) may receive prophylactic treatment.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration. In an embodiment, the administration is intravenous and/or oral.

In combination therapy, the dosage and frequency of administration of each component of the combination can be controlled independently. For example, one compound may be administered three times per day, while the second compound may be administered once per day. Combination therapy may be given in on-and-off cycles that include rest periods so that the subject's body has a chance to recover from any as yet unforeseen side effects. The compounds may also be formulated together such that one administration delivers both compounds.

The administration of a combination or composition as described herein (e.g., compound of formula (I) or a pharmaceutically acceptable salt or derivative thereof/vancomycin or derivative thereof combination) may be by any suitable means that results in an amount sufficient to treat a Gram-positive bacterial infection or an amount effective to reduce bacterial growth at a target site. A compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each agent of the combination or composition may be formulated in a variety of ways that are known in the art. For example, the first and second agents may be formulated together or separately. Desirably, the first and second agents are formulated together for the simultaneous or near simultaneous administration of the agents.

The term “treatment” as used herein covers any treatment of a condition or disease in a subject, preferably a mammal, more preferably a human, and includes: (i) preventing the disease or condition from occurring in a subject which may be predisposed to the disease but has not yet been diagnosed as having it; (ii) inhibiting the disease or condition, i.e., arresting its development; (iii) relieving the disease or condition, i.e., causing regression of the condition; or (iv) relieving the conditions caused by the disease, i.e., symptoms of the disease.

The term “preventing” as used herein refers to administering a medicament beforehand to avert or forestall the appearance of one or more symptoms of a disease or disorder. The person of ordinary skill in the medical art recognizes that the term “prevent” is not an absolute term. In the medical art it is understood to refer to the prophylactic administration of a drug to substantially diminish the likelihood or seriousness of a condition, or symptom of the condition and this is the sense intended in this disclosure. As used in a standard text in the field, the Physician's Desk Reference, the terms “prevent,” “preventing,” and “prevention,” with regard to a disorder or disease, refer to averting the cause, effects, symptoms or progression of a disease or disorder prior to the disease or disorder fully manifesting itself.

All patents, patent applications and publications mentioned herein are hereby incorporated by reference in their entireties.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

### Example 1—Interaction of Bdm-I and Vancomycin with MRSA

Isolate MIC Testing and Statistical Analysis

One-hundred and three MRSA bacteraemia isolates (from Liverpool Hospital, Sydney, Australia) were selected for this study. BDM-I MICs were determined by the broth microdilution method (BMD) as described by the CLSI (Clinical Laboratory Standards Institute, Methods for Dilution Antimicrobial Susceptibility Tests for Bacteria That Grow Aerobically: Ninth Edition: Approved Standard M07-A9, CLSI, Wayne, Pa., USA, 2012) using CAMHB (Oxoid-Thermo Fisher, Hampshire, UK). BDM-I concentrations ranged from 1 to 10 mg/L in 1 mg/L increments. All isolates had previously undergone vancomycin MIC testing and hVISA/VISA characterization (van Hal, Barbagiannakos et al., 2011; van Hal, Jones et al., 2011). Statistical analysis to determine correlation between variables was completed using the Spearman's test; with P<0.05 considered significant. Calculations were completed using SPSS statistical software (version 22.0; SPSS Inc, Chicago, Ill., USA).

In Vitro Mutant Generation

Induction experiments were completed on two MRSA isolates, Sa057 (VSSA) and Sa375 (VISA) in triplicate series over a period of approximately 110 days. Isolates were passaged daily in Luria Bertani broth (LBB) supplemented with increasing concentrations of BDM-I during this time, with isolates stored for further analysis at −80° C. as the LBB BDM-I concentration was increased.

Whole Genome Sequencing

DNA libraries were constructed using genomic DNA extracted with the ISOLATE Genomic DNA extraction kit (Bioline, London, ENGLAND) as per the manufacturer's instructions. Using the NEBNext Fast DNA Fragmentation & Library Prep Set for Ion Torrent (New England Biolabs, Ipswich USA) and the Ion Xpress Plus fragment library kit (Life Technologies, Carlsbad, Calif., USA), 400-bp barcoded libraries were generated as per the manufacturer's instructions. The barcoded libraries were then amplified using a thermal cycler and subsequently purified using the Agencourt AMPure XP Reagent (Beckman Coulter, Brea, Calif., USA). The purified libraries were then combined together and bound to Ion Sphere™ Particles for enrichment and clonal amplification using an Ion OneTouch™ 2 System (Life Technologies) as per the manufacturer's instructions. Quantification of the amplified library was completed using a Qubit 2.0 Fluorometer (Life Technologies). The DNA samples were loaded onto an Ion 318™ v2 chip (Life Technologies) and sequenced in accordance to the manufacturer's instructions. Subsequently, whole genome sequencing reads were analysed using CLC Genomics Workbench ver.7.0.3 (CLCbio, N Aarhus DEN).

Electron Microscopy of Sa375 Mutants

Transmission electron microscopy (TEM) was completed on Sa375 mutants following whole genome sequencing. Overnight cultures of each mutant Sa375 isolate as well as the progenitor Sa375 and Sa057 isolates were centrifuged and re-suspended in a 2.5% Glutaraldehyde solution in 0.1M cacodylate buffer (pH 7.4). Following fixation for at least 4 hr, the buffer was exchanged with 2% Osmium tetroxide and rinsed with Sodium acetate, and finally stained with Uranyl acetate for 60 min. The samples were then dehydrated in alcohol then infiltrated with Spurr resin in acetone (1:1) for 30 min, then again in 6:1 resin for 22 hr. Polymerisation of samples was then allowed to carry out for 15 hr at 70° C. before being cut and imaged using a Morgagni 268D transmission electron microscope (FEI, Eindhoven, THE NETHERLANDS) used at 80 kV and fitted with a Soft Imaging Systems MegaView III CCD camera (Munster, GERMANY). In total, ten measurements were taken on ten individual cells.

Results

The BDM-I MICs for a collection of clinical MRSA isolates, with varying vancomycin susceptibilities, were determined using the BMD method. MICs ranged from 2-5 mg/L and the average values for VSSA (n=43), hVISA (n=54) and VISA isolates were 3.42, 3.28 and 2.5 mg/L, respectively (Table 1). Interestingly, when compared to previously determined vancomycin MICs for these isolates, a ‘see-saw’ effect was observed, as BDM-I MICs were inversely correlated (Rho=−0.24; P=0.0145). This is best illustrated in Table 1, where VSSA isolates have a higher average BDM-I MIC in comparison to that for hVISA and VISA isolates.

TABLE 1
Average BDM-I and vancomycin MICs and MIC₅₀ values
as determined by BMD, with isolates grouped according
to vancomycin phenotype. Of note is the inverse correlation
between BDM-I and vancomycin MICs.

| | Vancomycin BDM-I | Vancomycin |
|---|---|---|
| Isolate | MIC (mg/L) | MIC (mg/L) |

| | | | | |
|---|---|---|---|---|
| Phenotype | MIC₅₀ | MIC | MIC₅₀ | MIC |

| | | | | | |
|---|---|---|---|---|---|
| VSSA | (n = 43) | 1.71 | 3.42 | 0.58 | 1.16 |
| hVISA | (n = 54) | 1.64 | 3.28 | 0.96 | 1.92 |
| VISA | (n = 6) | 1.25 | 2.5 | 1.1 | 2.2 |

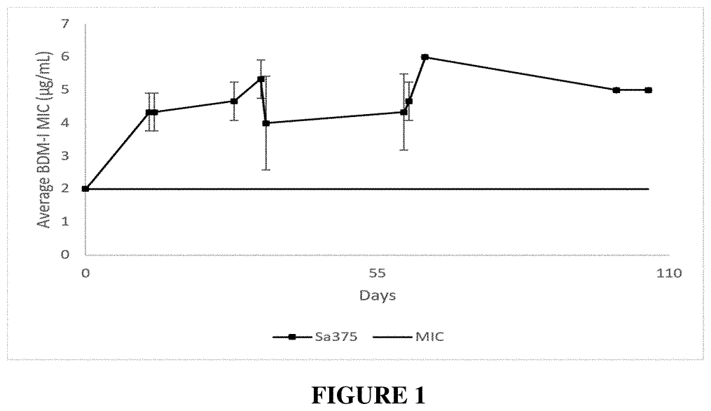

In order to further explore the antimicrobial potential of BDM-I and the observed inverse relationship between BDM-I and vancomycin MICs, the inventors assessed the ability of MRSA isolates (selected from the above collection) to develop increased BDM-I MICs during an extended period of exposure; two MRSA isolates, Sa057 (VSSA) and Sa375 (VISA), were subcultured (in triplicate) in the presence of increasing concentrations of BDM-I for approximately 110 days (see FIG. 1); only the graph for Sa375 is shown. In a clinical context such information is important, as the prolonged use of antibiotics in vivo is often associated with treatment failure due to the generation of resistant mutants (Mwangi, Wu et al., 104; Pelegi, Miyakis et al., 2012).

MIC testing of the triplicate day 110 cultures revealed that isolated Sa057 (VSSA) colonies inconsistently displayed small BDM-I MIC increases of 1.5 mg/L (i.e., increased from 3.5 to 5 mg/L); note that only 6 out of 21 colonies tested across the triplicate experiments displayed this small increase. In contrast, all Sa375 (VISA) colonies tested (21 out of 21) displayed a BDM-I MIC increase of 3 mg/L (i.e., increased from 2 to 5 mg/L).

Subsequently, in order to identify mutations associated with this MIC increase, whole genome sequencing (WGS) was performed on genomic DNA isolated from 9 (3 from each series) Sa375 mutant colonies. Interestingly, following variant analysis, single nucleotide polymorphisms (SNPs) were identified (for all colonies) within the walK gene, which encodes a multi-sensor signal transduction histidine kinase (see Table 2). It is important to note that Sa375 already contained a mutation within walK (amino acid change M220I) that resulted in an increase in cell wall thickness (van Hal, Steen et al., 2014), which is the phenotype associated with intermediate vancomycin resistance. As such, due to the observed inverse relationship between BDM-I and vancomycin MICs, it was hypothesized that the newly identified walK mutations were compensatory and resulted in a decrease in cell wall thickness and thus increased sensitivity to vancomycin.

TABLE 2
MUTATIONS IDENTIFIED WITHIN SEQUENCED COLONIES
WITH INCREASED BDM-I MICS. THE MUTATIONS SHOWN
ARE THOSE WHICH ARE PRESENT WITHIN ALL COLONIES
SEQUENCED FOR BOTH SA057 AND SA375

| Isolate | Series/Colony | Region | Mutation | AA Change | Gene |
|---|---|---|---|---|---|
| Sa375 | 1/* | 25210 | C → T | Leu37Phe | walK |
| | 2/* | 26779 | G → A | Gly560Ser | walK |
| | 3/* | 25189 | G → T | Gly30Trp | walK |

*Mutation identified within all three colonies of the series.

To confirm this, select Sa375 mutants (1 from each series) and controls (Sa057 and Sa375) were subjected to transmission electron microscopy (TEM) and vancomycin MIC testing.

In summary, each mutant displayed a reduction in cell wall thickness in comparison to the Sa375 progenitor strain. Additional MIC testing also revealed that the mutants were now susceptible to vancomycin. Vancomycin MICs for the mutant isolates were 0.5 mg/L for Sa375-L37F and Sa375-G560S, and 1 mg/L for Sa375-G30W, which are significantly lower than the Sa375 progenitor vancomycin MIC of 4 mg/L. This data suggests that reverting to a VSSA phenotype with a thinner cell wall is beneficial in the presence of BDM-I, but results in an increased susceptibility to vancomycin.

Conclusion

Based on the above results, BDM-I shows potential as an option for salvage therapy in the context of reduced-vancomycin-susceptible MRSA infections, as the associated phenotype (i.e., increased cell wall thickness) results in increased BDM-I sensitivity.

### Example 2—BDM-I Synergy Studies

BDM-I synergism was determined using the checkerboard method as described in the literature (Orha, Bayram et al., 2005; Sopirala, Mangino et al., 2010). Antibiotic dilutions were prepared in MHB at double the desired concentration and combined in equal volumes (50 μL) into a single well of a sterile 96-well plate. The final concentration range for each antibiotic was as follows; 5-0.25 mg/L BDM-I, and two-fold dilutions of vancomycin from 16-0.0625 mg/L. 10 μL of the prepared bacterial suspension (diluted to 1×10⁵ Cfu) was then inoculated into each well, and the plate then incubated at 37° C. for 16-20 hours overnight. MIC values were then determined as described by the CLSI for broth microdilution testing. Determination of the fractional inhibitory concentration index (FICI) was calculated with the equation: FICI=FIC A+FIC B, where FIC A is the MIC of drug A in combination divided by the MIC of drug A alone, and FIC B is the same for drug B. FICI values were then determined as follows: FICI of ≤0.5 indicates synergy, FICI of >0.5 to ≤4 indicates no interaction, and FICI of >4 indicates antagonism (Odds, 2003).

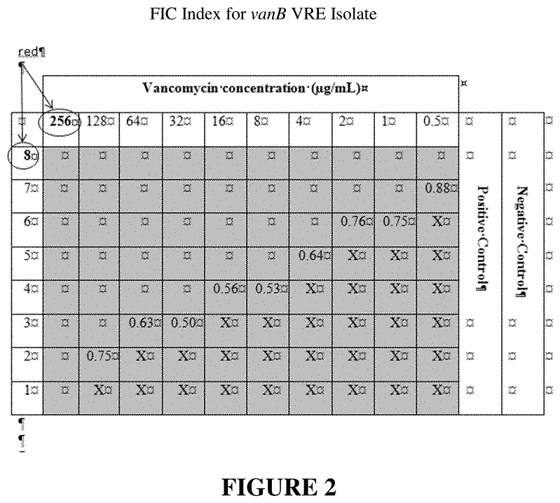

FIG. 2 shows that vancomycin combined with BDM-I at fixed concentrations demonstrated synergistic activity a vanB VRE isolate.

### REFERENCES

The following references, to the extent that they provide exemplary procedural or other details supplementary to those set forth herein, are specifically incorporated herein in their entirety by express reference thereto:
Boucher, H W, “Challenges in anti-infective development in the era of bad bugs, no drugs: A regulatory perspective using the example of bloodstream infection as an indication,” Clin. Infect. Dis., 50:S4-S9 (2010).
Clinical Laboratory Standards Institute, Methods for Dilution Antimicrobial Susceptibility Tests for Bacteria That Grow Aerobically: Ninth Edition: Approved Standard M07-A9, CLSI, Wayne, Pa., USA (2012).
Denisenko, P P, Sapronov, N S, Tarasenko, A A, “Antimicrobial and radioprotective compounds,” PCT Intl. Pat. Publ. No. WO/2002/102789 (Mar. 27, 2002).
Fridkin, S K et al., “Epidemiological and microbiological characterization of infections caused by Staphylococcus aureus with reduced susceptibility to vancomycin, United States, 1997-2001,” Clin. Infect. Dis., 36(4):429-439 (2003).
Hiramatsu, K, Aritaka, N, Hanaki, H, et al., Dissemination in Japanese hospitals of strains of Staphylococcus aureus heterogeneously resistant to vancomycin,” Lancet, 350: 1670-1673 (1997).
Hiramatsu, K, Hanaki, H, Ino, T, Yabuta, K, Oguri, T, Tenover, F C, “Methicillin-resistant Staphylococcus aureus clinical strain with reduced vancomycin susceptibility,” J. Antimicrob. Chemother., 40(1): 135-136 (1997).
Howden, B P, Davies, J K, Johnson, P D, Stinear, T P, and Grayson, M L, “Reduced vancomycin susceptibility in Staphylococcus aureus, including vancomycin-intermediate and heterogeneous vancomycin-intermediate strains: resistance mechanisms, laboratory detection, and clinical implications,” Clin. Microbiol. Rev., 23:99-139 (2010).
Linares, J, “The VISA/GISA problem: therapeutic implications,” Clin. Microbiol. Infect., 7(suppl 4):8-15 (2001).
Milhazes, N, Calheiros, R, Marques, MPM, Garrido, J, Cordeiro, MNDS, Rodrigues, C, Quinteira, S, Novais, C, Peixe, L, Borges, F, 13-Nitrostyrene derivatives as potential antibacterial agents: A structure-property-activity relationship study,” Bioorg. Med. Chem., 14:4078-4088 (2006).
Mwangi, M M, Wu, S W, Zhou, Y et al., “Tracking the in vivo evolution of multidrug resistance in Staphylococcus aureus by whole-genome sequencing,” Proc. Nat'l Acad. Sci. USA, 104:9451-9456 (2007).
Nicoletti, A, Borland, R, Williams, T G, “Growth promotion method,” PCT Intl. Pat. App. Publ. No. PCT/AU2004/000253 (27 Feb. 2004).
Nicoletti, G, Cornell, H J, Hugel, H M, White, K S, Nguyen, T, Zalizniak, L,
Nugegoda, D, “Synthesis and antimicrobial activity of nitroalkenyl arenes,” Anti-Infect. Agents, 11:179-191 (2013).
Odds, F C, “Synergy, antagonism, and what the chequerboard puts between them,” J. Antimicrobial Chemotherapy, 52:1 (2003).
Orhan, G, Bayram, A, Zer, Y et al., “Synergy tests by E test and checkerboard methods of antimicrobial combinations against Brucella melitensis,” J. Clinical Microbiology, 43:140-143 (2005).
Park, J, Pei, D, “trans-β-Nitrostyrene derivatives as slow-binding Inhibitors of protein tyrosine phosphatases,” Biochemistry, 43:15014-15021 (2004).
Payne, D J, Gwynn, M N, Holmes, D J, Pompliano, D L, “Drugs for bad bugs: confronting the challenges of antibacterial discovery,” Nat. Rev. Drug Discov., 6:29-40 (2007).
Peleg, A Y, Miyakis, S, Ward, D V et al., “Whole genome characterization of the mechanisms of daptomycin resistance in clinical and laboratory derived isolates of Staphylococcus aureus,” PLoS One, 7:e28316 (2012).
Perkins, H R, “Vancomycin and related antibiotics,” Pharmacol. Ther., 16:181-197 (1982).
Rice, L B, “Federal funding for the study of antimicrobial resistance in nosocomial pathogens: no ESKAPE,” J. Infect. Dis., 197(8):1079-1081 (2008).
Schales, O, Graefe, H A, “Arylnitroalkenes: a new group of antibacterial agents,” J. Am. Chem. Soc., 74:4486-4490 (1952).
Sievert, D M, Rudrik, J T, Patel, J B, McDonald, L C, Wilkins, M J, Hageman, J C, “Vancomycin-resistant Staphylococcus aureus in the United States, 2002-2006,” Clin. Infect. Dis., 46(5):668-674 (2008).
Sopirala, M M, Mangino, J E, Gebreyes, W A et al., “Synergy testing by Etest, microdilution checkerboard, and time-kill methods for pan-drug-resistant Acinetobacter baumannii,” Antimicrobial Agents Chemotherapy, 54:4678-4683 (2010).
Van Hal, S J, Barbagiannakos, T, Jones, M et al., “Methicillin-resistant Staphylococcus aureus vancomycin susceptibility testing: methodology correlations, temporal trends and clonal patterns,” J. Antimicrobial Chemotherapy, 66:2284-2287 (2011).
Van Hal, S J, Jones, M, Gosbell, I B et al., “Vancomycin heteroresistance is associated with reduced mortality in ST239 methicillin-resistant Staphylococcus aureus blood stream infections,” PLoS One, 6:e21217 (2011).
Van Hal, S J, Steen, J A, Espedido, B A et al., “In vivo evolution of antimicrobial resistance in a series of Staphylococcus aureus patient isolates: the entire picture or a cautionary tale,” J. Antimicrobial Chemotherapy, 69:363-367 (2014).
Vincent, C, Duclos, B, Grangeasse, C, Vaganay, E, Riberty, M, Cozzone, A J,
Doublet, P, “Relationship between exopolysaccharide production and protein-tyrosine phosphorylation in Gram-negative bacteria,” J. Mol. Biol., 304:311-321 (2000).
White, K S, “The antimicrobial mechanism of action of 3,4-methylenedioxy-β-nitropropene,” Ph.D. Thesis. RMIT University: Melbourne, AUSTRALIA (2008).
Worthen, L. R.; Bond, H. W. Antimicrobial activity of some betanitrostyrenes,” J. Pharm. Sci., 59:1185-1186 (1970).
Zhu, W, Murray, P R, and Huskins, W C, “Dissemination of an Enterococcus Inc 18-like VanA plasmid associated with vancomycin-resistant Staphylococcus aureus,” Antimicrob. Agents Chemother., 54:4214-4320 (2010).

What is claimed is:

1. A method of treating S. aureus infection in a patient wherein the S. aureus has at least partial resistance to vancomycin, comprising: administering to the patent an effective amount of a compound of formula (I) or a pharmaceutically-acceptable salt or derivative thereof: 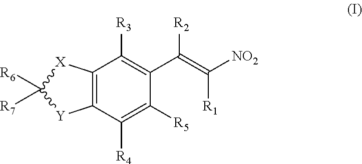
in which
X and Y are either the same or different and are each a heteroatom selected from the group consisting of 0, N, and S; [symbol]
is a double or single bond depending on the heteroatoms X and Y;
R₁ to R₅ are either the same or different, and selected from hydrogen, alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, arylthio, acylthio or phosphorus-containing compounds; and
R₆ and R₇ are either the same or different, and selected from hydrogen, alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, arylthio, acylthio or phosphorus-containing compounds, or one of R₆ and R₇ is absent when there is a double bond present.

2. A method of treating a S aureus infection in a patient in which the infection has failed to resolve following vancomycin treatment, comprising: administering to the patient a compound of formula (I) or a pharmaceutically-acceptable salt or derivative thereof: 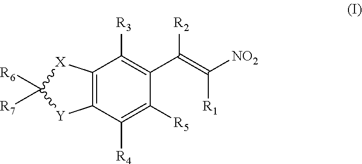
in which
X and Y are either the same or different and are each a heteroatom selected from the group consisting of O, N, and S; [symbol]
is a double or single bond depending on the heteroatoms X and Y;
R₁ to R₅ are either the same or different and selected from hydrogen, alkyl, alkenyl, alkynyl, aryl, halo, haloalkyl, haloalkenyl, haloalkynyl, haloaryl, hydroxy, alkoxy, alkenyloxy, acyloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, arylthio, acylthio or phosphorus-containing compounds; and
R₆ and R₇ are either the same or different, and selected from hydrogen, alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, arylthio, acylthio or phosphorus-containing compounds, or one of R₆ and R₇ is absent when there is a double bond present.

3. The method of claim 1 or claim 2, wherein X and Y are either the same or different, and are selected from O and N.

4. The method of claim 3, wherein both X and Y are oxygen.

5. The method of claim 1, wherein R₁ and R₂ are either the same or different, and are selected from the group consisting of hydrogen, hydroxy, halogen and optionally substituted C₁₋₆ alkyl.

6. The method of claim 1, wherein R₃ to R₅ are either the same or different, and are selected from the group consisting of hydrogen, hydroxy, halogen, nitro, C₁₋₆ alkoxy, and optionally substituted C₁₋₆ alkyl.

7. The method of claim 1, wherein the compound of formula I is selected from the group consisting of:
3,4-methylenedioxy-β-methyl-β-nitrostyrene, 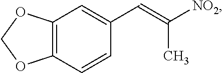
(wherein X and Y are O, R₁ is methyl, and R₂ and R₃ are hydrogen);
3,4-methylenedioxy-β-nitrostyrene, 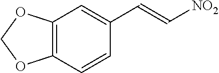 [SMILES: O=[N+]([O-])/C=C/c1ccc2c(c1)OCO2]
(wherein X and Y are O, and R₁ to R₃ are hydrogen);
benzimidazole-5-β-nitropropylene, 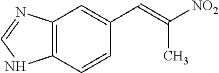
(wherein X is N, Y is NH, R₁ is methyl, and R₂ and R₃ are hydrogen);
2-methyl benzimidazole-5-β-nitroethylene, 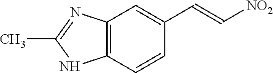
(wherein X is N, Y is NH, R₁ is hydrogen, R₂ is methyl, and R₃ is absent);
benzoxazole-5-β-nitroethylene, 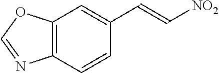 [SMILES: O=[N+]([O-])/C=C/c1ccc2ncoc2c1]
(wherein X is O, Y is N, R₁ and R₂ are hydrogen, and R₃ is absent); and
2-methyl benzoxazole-5-β-nitropropylene, 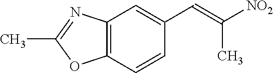 [SMILES: C/C(=C\c1ccc2oc(C)nc2c1)[N+](=O)[O-]]
(wherein X is N, Y is O, R₁ and R₂ are methyl, and R₃ is absent).

8. The method of claim 7, wherein the compound of formula I is 3,4-methylenedioxy-β-methyl-β-nitrostyrene (BDM-I): 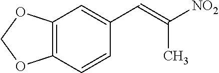
(i.e., wherein X and Y are O, R₁ is methyl, and R₂ and R₃ are hydrogen).

9. The method of claim 1, wherein the S. aureus is VRSA, VISA or hVISA.

10. A method of treating an Enterococcal infection in a patient, comprising: administering to the patient vancomycin, or a derivative thereof; and a compound of formula (I), or a pharmaceutically-acceptable salt or derivative thereof: 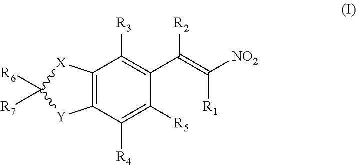
in which
X and Y are either the same or different and are each a heteroatom selected from the group consisting of O, N, and S; [symbol]
is a double or single bond depending on the heteroatoms X and Y;
R₁ to R₅ are either the same or different and selected from hydrogen, alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, acylthio, acylthio or phosphorus-containing compounds; and
R₆ and R₇ are either the same or different, and selected from hydrogen, alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, arylthio, acylthio or phosphorus-containing compounds, or one of R₆ and R₇ is absent when there is a double bond present.

11. The method of claim 10, wherein X and Y are either the same or different, and are selected from O and N.

12. The method of claim 11, wherein both X and Y are oxygen.

13. The method of claim 10, wherein R₁ and R₂ are either the same or different, and selected from hydrogen, hydroxy, halogen or optionally substituted C₁₋₆ alkyl.

14. The method of claim 10, wherein R₃ to R₅ are either the same or different, and are selected from the group consisting of hydrogen, hydroxy, halogen, nitro, C₁₋₆ alkoxy, and optionally substituted C₁₋₆ alkyl.

15. The method of claim 10, wherein the compound of formula I is selected from the group consisting of: 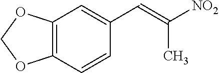
3,4-methylenedioxy-β-methyl-β-nitrostyrene (BDM-I), 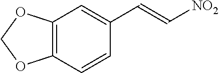 [SMILES: O=[N+]([O-])/C=C/c1ccc2c(c1)OCO2]
3,4-methylenedioxy-β-nitrostyrene, 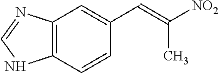
benzimidazole-5-β-nitropropylene, 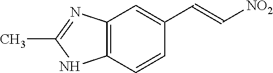
2-methyl benzimidazole-5-β-nitroethylene, 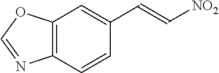 [SMILES: O=[N+]([O-])/C=C/c1ccc2ncoc2c1]
benzoxazole-5-β-nitroethylene, and 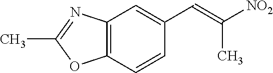 [SMILES: C/C(=C\c1ccc2oc(C)nc2c1)[N+](=O)[O-]]
2-methyl benzoxazole-5-β-nitropropylene.

16. The method of claim 15, in which X and Y are 0, R₁ is methyl, and R₂ and R₃ are hydrogen: 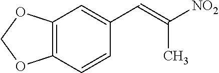
(3,4-methylenedioxy-β-methyl-β-nitrostyrene; BDM-I).

17. The method of claim 10, wherein the Enterococcus is vanB VRE.

18. The method of claim 17, wherein the Enterococcus is E. faecalis or E. faecium.

19. A composition comprising a combination of vancomycin or a derivative thereof and a compound of formula I, or a pharmaceutically-acceptable salt or derivative thereof, as defined herein.
